(19) United States
(12) Patent Application Publication
(10) Pub. No.: US 2003/0220415 A1
(43) Pub. Date: Nov. 27, 2003

(54) Heterocyclic amine diol compounds and their biocidal derivatives

(75) Inventors: Shelby D. Worley, Auburn, AL (US); Yanjun Li, Kirkland, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Auburn University and Vanson Halosource, Inc.

(21) Appl. No.: 10/190,897

(22) Filed: Jul. 5, 2002

Related U.S. Application Data
(60) Provisional application No. 60/379,969, filed on May 10, 2002.

(51) Int. Cl. C08G 18/28; C08G 18/77; C07D 233/40

(52) U.S. Cl. 523/122; 528/73; 548/319.1

(57) ABSTRACT

Heterocyclic amine polyol compounds and methods for making the compounds and polymers for coatings and materials that can be rendered biocidal by exposure to halogen solutions. One embodiment of compounds have the formula:

wherein R₁ and R₂ are at least one of an alkyl group of 1 to 6 carbons or phenyl group;

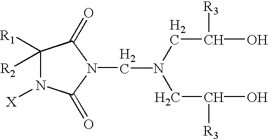

R₃ is at least one of a hydrogen and an alkyl group of 1 to 6 carbons; and X is at least one of a hydrogen and a halogen. The compounds are rendered biocidal when X is a halogen. The compounds are useful for making protective films, coatings, sealants, adhesives, sponges, foams, paints and in other applications.

### CROSS-REFERENCE TO RELATED APPLICATION

This application claims the benefit of provisional U.S. Application No. 60/379,969, filed May 10, 2002, incorporated herein by reference in its entirety.

### FIELD OF THE INVENTION

The present invention relates to heterocyclic amine polyol compounds that are rendered biocidal upon halogenation and are used in protective films, coatings, sealants, adhesives, sponges, foams, paints and other applications.

### BACKGROUND OF THE INVENTION

Previous attempts to incorporate biocidal activity into materials and coatings have primarily involved two methods. In the first, biocides are physically mixed or blended into the materials and coatings. In the second, biocidal functional groups are chemically bonded to polymers or copolymers in the materials and coatings. For example, U.S. Pat. Nos. 5,902,818; 6,162,452, and U.S. application Ser. No. 09/710,090, each incorporated herein by reference in its entirety, disclose surface active N-halamines. Chemical binding is preferred for long-term biocidal activity, as long as the bound biocidal functionality does not adversely affect the other desired properties of the material, such as strength, appearance, and chemical resistivity.

Weak biocides, such as polyquaternary ammonium salts, have been introduced into the porous structure of sponges. Disinfectant dyes have been bound to sponges. The sponges exhibit biocidal activity, but the contact time required to produce biocidal action is undesirably long. This is especially true for gram negative bacteria like Escherichia coli.

Anti-fouling polyurethanes have been prepared by chemical incorporation of tributyl tin and quaternary ammonium salts. Unfortunately, coatings containing organotin compounds are threats to the environment, and polyquaternary ammonium salts are weak biocides that are nonregenerable.

A new class of biocidal monomers and polymers known as N-halamines has recently been developed. A non-toxic, non-irritating, and cost-effective material, poly-1,3-dichloro-5-methyl-5-(4′-vinylphenyl)hydantoin, is an inexpensive derivative of polystyrene. It was first described in U.S. Pat. No. 5,490,983, issued to Worley et al., incorporated herein by reference in its entirety. This polymer is effective against a broad spectrum of pathogens including Staphylococcus aureus, Pseudomonas aeruginosa, Escherichia coli, Candida albicans, Klebsiella terrigena, poliovirus, and rotavirus, reducing the required contact times.

Heterocyclic functional groups such as hydantoins, oxazolidinones, and imidazolidinones have also been employed recently using grafting procedures in producing biocidal cellulose (U.S. Pat. No. 5,882,357), biocidal films on surfaces (U.S. Pat. No. 5,902,818), biocidal NYLON (U.S. patent application Ser. No. 09/615,184), and biocidal polyester (U.S. patent application Ser. No. 09/866,535). Each of these patents and patent applications is incorporated herein by reference in its entirety.

### SUMMARY OF THE INVENTION

The present invention is directed to a compound having a heterocyclic amine group covalently bonded through a linking group, for example, an alkylene group of 1 to 6 carbons, to an amine polyol group. A preferred alkylene group is methylene and a preferred amine polyol is diethanolamine. Preferably, the amine polyol is a diol. Examples of suitable heterocyclic amines for use in the invention are hydantoins, imidazolidinones, oxazolidinones, glycolurils, isocyanurates, and triazinediones. The polyol group is reactive with other functional groups. For example, polyols react with isocyanates to form polyurethanes. Polyurethanes are used in many ways. Polyurethanes can be rendered biocidal by incorporating compounds of the present invention.

In one embodiment, the compound of the invention is represented by the formula:

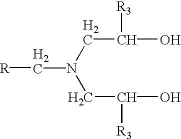

wherein R represents a heterocyclic amine selected from at least one of a hydantoin, an imidazolidinone, an oxazolidinone, an isocyanurate, a glycoluril, or triazinedione; and R₃ is hydrogen or an alkyl group of 1 to 6 carbons. The two R₃ groups can be the same or different. Hydantoins, imidazolidinones, isocyanrates, glycolurils, and triazinediones are bound to the amine polyol through a linking group. The binding site for these heterocylic amines is a nitrogen heteroatom. Oxazolidinones are bound to the amine polyol through a linking group. The binding site for oxazolidinone is a carbon atom, to leave a nitrogen heteroatom available to bind with a halogen.

A linking group, for example an alkylene group, such as methylene, links the heterocyclic amine group to the polyol group. The R group (i.e., heterocyclic amine) also has a binding site for a halogen. Halogenation renders the compound biocidal. In another embodiment, the compound is represented by the formula:

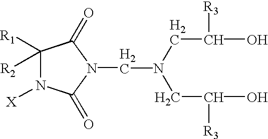

wherein R₁ and R₂ are selected from an alkyl group of from 1 to 6 carbons and phenyl group; R₃ is selected from hydrogen and an alkyl group of from 1 to 6 carbons; and X is selected from hydrogen and a halogen. Compounds in which X is a halogen are biocidally active. Suitable halogens include chlorine and bromine. The two R₃ groups can be the same or different.

A heterocyclic amine polyol polymer is made from the compounds of the present invention by reacting the compounds with at least one other unit having a functional group that is reactive toward an alcohol. The units can be individual units or units that are incorporated into a polymer. When the heterocyclic amine is halogenated, the polymers are biocidal polymers. Protective films can be made by forming a thin layer, and allowing the thin layer to cure.

In one embodiment, a polymer includes moieties with the formula:

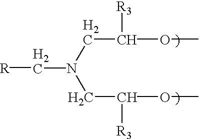

wherein R is a heterocyclic amine selected from a hydantoin, an imidazolidinone, an oxazolidnone, an isocyanurate, a glycoluril, or triazinedione. R₃ is hydrogen or an alkyl group of 1 to 6 carbons. The two R₃ groups can be the same or different.

In another embodiment, a polymer has moieties with the formula:

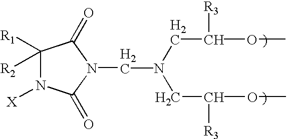

wherein R₁ and R₂ are selected from an alkyl group of 1 to 6 carbons or phenyl group. R₃ is hydrogen or an alkyl group of 1 to 6 carbons. The two R₃ groups can be the same or different. X is hydrogen or a halogen.

In one embodiment, a paint composition is formed by mixing a heterocyclic amine polyol of the invention, a second polyol, such as an acrylic polyol, with a paint component having isocyanate groups. The resulting urethane group formed from the alcohol group of the heterocyclic amine polyol and the isocyanate group of the paint component covalently bonds the heterocyclic amine group through the amine polyol group to the paint component. The heterocyclic amine group has at least one site that is reactive with a halogen, rendering biocidal functionality to the paint composition on halogenation of the site. In addition, any composition containing units with functional groups that are reactive toward an alcohol group can be mixed with the heterocyclic amine polyol compounds to produce heterocyclic amine polyol polymers that can be rendered biocidal upon halogenation of the cyclic amine.

The biocidal polymers can be used to inactivate pathogenic microorganisms such as bacteria, fungi, and yeasts, as well as virus particles, which can cause infectious diseases, and those microorganisms which cause noxious odors and unpleasant coloring such as mildew. In one embodiment, the biocidal polymers can be made into coatings. In other embodiments, the polymers are used to make consumer articles.

A marked advantage of the invention over prior technology is that the compounds and polymers are much more effective against pathogenic microorganisms such as S. aureus and P. aeruginosa, typically encountered in medical applications, than are commercial biocides such as the quaternary ammonium salts. The biocidal polymers of the present invention can inactivate disease-causing pathogens and odor-causing microorganisms. For this reason, biocidal polymers of the present invention will be particularly advantageous for widespread use in medical settings such as hospitals, nursing facilities, and research laboratories. These biocidal polymers will also be useful for making biocidal coatings and materials such as sponges, paints, and foams, and in a variety of other industrial settings or for the home.

### DETAILED DESCRIPTION OF THE PREFERRED EMBODIMENT

The present invention is directed to a compound having a heterocyclic amine functional group covalently bonded through a linking group, for example, an alkylene group, such as methylene, to an amine polyol functional group, wherein the heterocyclic amine has at least one site that is reactive toward a halogen. When halogenated, the compound is biocidal. In one embodiment, the reactive site is a nitrogen heteroatom. Examples of suitable heterocyclic amines include hydantoins having structures I or II; imidazolidinones having structures III, IV, or V; oxazolidinones having structure VI; glycolurils having structure VII; isocyanurates having structure VIII; and triazinediones having structure IX. Examples of all the above heterocyclic amines are shown below, wherein L depicts one suitable site for a linking and polyol group; and R₄-R₇ are alkyl groups. For structure VI, however, R₄ can be ethyl or hydroxymethyl. X can be hydrogen in the non-biocidal derivative of the compounds or a halogen in the biocidal derivative of the compounds.

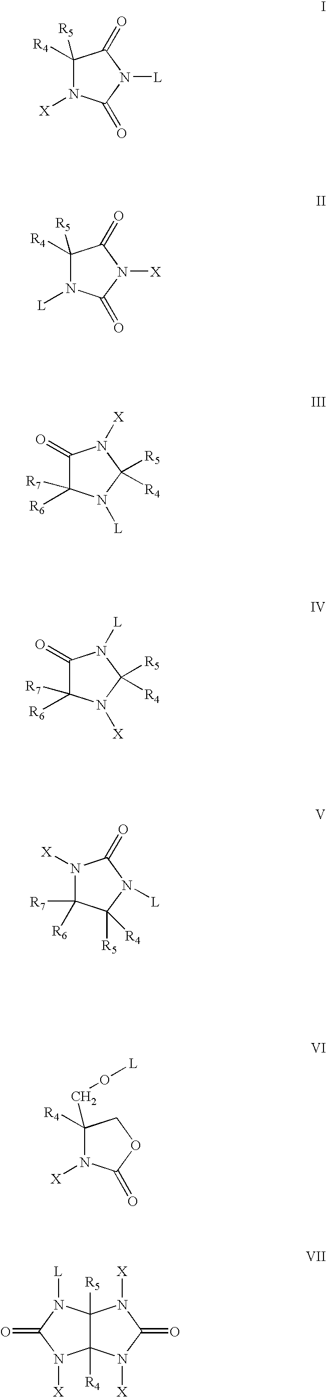

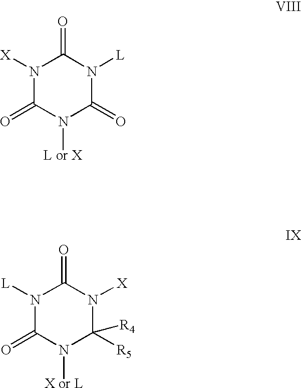

One embodiment of a compound made according to the invention has the structure (X) shown below:

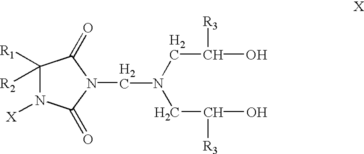

In this embodiment, R₁ and R₂ are alkyl groups of one to six carbons or phenyl groups, or any combination of these two groups. The alkyl groups may be linear or branched, substituted or unsubstituted. The phenyl groups may be phenyl or substituted phenyl. R₃ is hydrogen or an alkyl group of one to six carbons; and X is hydrogen or a halogen. The two R₃ groups can be the same or different.

The compound represented by structure (X) can be synthesized by reacting a 5,5-dialkylhydantoin with formaldehyde and then with an aminoalcohol. The reaction can proceed at ambient temperature. The reaction is exothermic. In a preferred embodiment, the 5,5-dialkylhydantoin is 5,5-dimethylhydantoin and the aminoalcohol is diethanolamine.

In an alternative method of making the compound represented by structure (X), a 3-hydroxyalkyl-5,5-dialkylhydantoin is reacted with an aminoalcohol at ambient temperature or higher. In a preferred embodiment, the 3-hydroxyalkyl-5,5-dialkylhydantoin is 3-hydroxymethyl-5,5-dimethylhydantoin and the aminoalcohol is diethanolamine. The purified compound is a white solid, which is very soluble in water.

The solvents used for these reactions can be aqueous or organic reagents compatible with water. Alcohols such as methanol are particularly useful, as the removal of excess water during purification is difficult. However, in the practical use of the compound in copolymerization reactions to form waterborne films, it is not necessary to remove all of the water because water may be an additive in the copolymerization. The reactants used in the synthesis of the compound of the invention are inexpensive and commercially available from vendors such as Aldrich Chemical Company (Milwaukee, Wis.), Fisher Scientific (Pittsburgh, Pa.), and TCI America (Portland, Oreg.).

In one embodiment, the compounds and polymers of the invention can be rendered biocidal by exposure to free halogen. The halogen bonds to a nitrogen heteroatom of the cyclic amine to produce biocidal activity. Suitable halogens include chlorine and bromine. Free chlorine can be provided by sources including, but not limited to, gaseous chlorine, sodium hypochlorite bleach, calcium hypochlorite, chloroisocyanurates, and dichlorohydantoins. In the case of dichlorohydantoins, the chlorine moiety on the imide nitrogen should transfer to the more stable amide nitrogen on the compound of the invention. Likewise, the compounds and polymers of the invention can be made biocidal by exposure to an aqueous solution of free bromine, including, but not limited to, molecular bromine liquid, sodium bromide in the presence of an oxidizer such as potassium peroxy monosulfate, and brominated hydantoins. Halogenation can also be effected in organic solvents employing free radical halogenating agents such as t-butyl hypochlorite.

The heterocyclic amine polyol compound or its biocidal halogenated derivative can be used to make rechargeable biocidal coatings and materials. In one embodiment, the compound of the invention can react with units that have functional groups that are reactive toward an alcohol. Suitable resins and polymers formed from compounds of the invention include epoxides, polyurethanes, polyureas, acrylics, vinyls, silanes, and siloxanes. In one embodiment, a polyurethane material is formed from the reaction of an isocyanate, an acrylic polyol, and a compound of the present invention. In one embodiment, the acrylic polyol and compound of the invention can be stored in a container as a mixture. A separate container holds the isocyanate. Upon mixing, the compounds will react yielding the polyurethane material. The polyurethane materials are used in sponges, foams, and paints, for example. By adjusting the proportions of the constituents that make up the polymers, various properties such as hardness, chemical resistivity, appearance, and biocidal activity can be controlled. If the unhalogenated compound of the invention is employed in producing a coating or material, exposure to aqueous free halogen after curing creates biocidal activity. In most cases, the coating or material will eventually lose its bound halogen, and hence its biocidal efficacy. The loss occurs through biocidal action, reaction with reducing agents, and slow evaporation. Nevertheless, the biocidal efficacy can be regenerated by subsequent exposure of the coating or material to additional free halogen.

An example of a biocidal waterborne acrylic polyurethane coating uses about 0.5 to about 1.0 gram (preferably about 0.7 gram) of the unhalogenated heterocyclic amine polyol, about 10.0 grams waterborne acrylic polyol, about 2.0 to about 3.4 grams (preferably about 2.45 grams) isocyanate, and about 1.4 to about 2.4 grams (preferably about 2.1 grams) of water. After thoroughly mixing, the components are spread onto a surface to form a thin film protective layer. The thin film is then allowed to cure at ambient temperature. The thin film can be chlorinated by exposure to aqueous, free chlorine, for example, from about 1 to about 100% CLOROX bleach, preferably about 10%, for about 0.5 to 3.0 hours, preferably about 1.0 hour. Following thorough rinsing with water and drying in air, the surface will be biocidal for at least 14 days. Regeneration of biocidal activity can be effected by further exposure to a charging solution including a halogen such as aqueous free chlorine. It is contemplated that the exposure to free chlorine on a large surface, such as a floor or wall, could be accomplished with a spraying device or sponging action, or by any suitable method.

In one embodiment, the compounds of the invention can be incorporated into paints that have constituents with isocyanate groups. Isocyanate groups can react with the hydroxyl groups in the compounds of the present invention to yield urethane groups; thus, bonding the heterocyclic amine group to the paint constituent. The hydantoin group, for example, has a site that can react with a halogen, such as chlorine or bromine, and in so doing will provide biocidal functionality to the paint composition. Halogenation can take place with suitable agents before or after application of the paint on a surface. A suitable amount of non-halogenated heterocyclic amine polyol compound in a paint composition ranges from about 1% to 30%, on a weight percent basis. In one embodiment, the paint composition includes about 5.3% by weight of heterocyclic amine polyol.

Rigid and flexible polyurethane foams can be produced from compounds of the invention. Additionally, polyurethane coatings, adhesives, sealants, and elastomers can also be prepared. Polyurethane coatings are mainly used in conjunction with aliphatic isocyanates and acrylic or polyester or polyols because of their outstanding ability to weather. For flexible elastomeric coatings, methylene bis-(4-cyclohexylisocyanate) (HMDI) and isophorone diisocyanate (IPDI) are used with polyester polyols, whereas higher functional derivatives of HDI and IPDI with acrylic polyols are mainly used in the formation of rigid coatings. Plastic coatings, textile coatings and artificial leather are based on either aliphatic or aromatic isocyanates. For light-stable textile coatings, combinations of isophoronediamine (as chain extender) are used. The polyurethane and/or polyurea coatings are applied either directly to the fabric or using transfer coating techniques. Microporous polyurethane sheets are used for shoe and textile applications. Polyurethane binder resins are also used to upgrade natural leather. Blocked aliphatic isocyanates or their derivatives are used for one-component coating systems. Masked polyols are also used for this application. Water-borne polyurethane coatings are formulated by incorporating ionic groups into the polymer backbone. These ionomers are dispersed in water through neutralization. Ionic polymers are also formulated from toluene-2,4-diisocyanate (TDI) and 4,4′-methylenediphenyl isocyanate (MDI). Polyurethane and polyurea ionomers are obtained from divalent metal salts of p-aminobenzoic acid, 4,4′-methylenedianiline (MDA), dialkylene glycol, and TDI. Polyurethane adhesives are known for their excellent adhesion, flexibility, toughness, high cohesive strength, and fast cure rates. Two-component adhesives consist of an isocyanate prepolymer, which is cured with low equivalent weight diols, polyols, diamines, or polyamines. Such systems can be used neat or as solution. The two components are kept separately before application.

Water-borne adhesives are preferred because of restrictions on the use of solvents. Low viscosity prepolymers are emulsified in water, followed by chain extension with water soluble glycols or diamines. As crosslinker polymeric MDI (PMDI) can be used. Waterborne polyurethane coatings are used for vacuum forming of polyvinylchloride sheeting to acrylonitrile butadiene styrene shelves.

Polyurethane sealant formulations use TDI or MDI prepolymers made from polyether polyols. The sealants contain 30 to 50 percent of the prepolymer; the remainder includes pigments, fillers, plasticizers, adhesion promoters, and other additives. Polyurethane elastomers are either thermoplastic or thermoset polymers, depending on the functionality of the monomers used. Thermoplastic polyurethane elastomers are segmented copolymers, comprising of hard and soft-segment blocks. The soft-segment blocks are formed from long-chain polyester or polyether polyols and MDI; the hard segments are formed from short chain diols, mainly 1,4-butanediol and MDI. Thermoset polyurethanes are crosslinked polymers which are produced by casting or reaction injection molding. Kirk-Othmer, Concise Encyclopedia of Chemical Technology, 4th ed., pages 2057-2060, are fully incorporated herein by reference.

The present invention is more particularly described in the following examples which are intended as illustrative only since numerous modifications and variations therein will be apparent to those skilled in the art.

### EXAMPLES

### Example 1

### Preparation of a Representative Unhalogenated Heterocyclic Amine Diol: 3-[1-bis(N,N-2-hydroxyethyl) aminomethyl]-5,5-dimethyl-1,3-imidazolidin-2,4-dione

A mixture of 400 mL of methanol, 132.1 g (1.0 mol) of 5,5-dimethylhydantoin (97% purity) and of 106.2 g (1.0 mol) of diethanolamine (99% purity) was added to a 11L round-bottom flask. Then 81.16 g (1.0 mol) of 37% formaldehyde solution was added dropwise to the mixture with stirring at ambient temperature. After the dropwise addition was completed, the reaction mixture was stirred for 2 additional hours at ambient temperature. The methanol solvent and water were then removed by vacuum evaporation. Two cycles of benzene addition (50 mL each) followed by vacuum evaporation were then employed to more completely remove water. The viscous residue produced was then dissolved in 300 mL of ethyl acetate, and 50 g of anhydrous sodium sulfate were added for further drying purposes. Following removal of the sodium sulfate by filtration, the solution was refrigerated. After 12 hours, a white solid product precipitated from the ethyl acetate solution. The product, which was removed by filtration from the cold solution, exhibited a melting point of 74-76° C., was produced in 61% yield; and was identified as 3-[1-bis(N,N-2-hydroxyethyl) aninomethyl]-5,5-dimethyl-1,3-imidazolidin-2,4-dione or 3-[1-bis(N,N-2-hydroxyethyl) aminomethyl]-5,5-dimethyl hydantoin. ¹H NMR (DMSO-d₆)δ1.28(6H), 2.65(4H), 3.40(4H), 4.31(2H), 4.39(2H), 8.28(1H); ¹³C NMR (DMSO-d₆)δ24.8, 54.5, 57.6, 57.8, 59.2, 156.2, 178.7; IR (KBr) 1295, 1346, 1439, 1710, 1764, 2814, 2974, 3227, 3474 cm⁻¹. The same product could be made as an aqueous slurry using water as the solvent without the presence of methanol in less pure form, the solid being recovered by drying in air in near quantitative yield.

### Example 2

### An Alternate Route to a Representative Unhalogenated Heterocyclic Amine Diol: 3-[1-bis(N,N-2-hydroxyethyl) aminomethyl]-5,5-dimethyl-1,3-imidazolidin-2,4-dione

A mixture of 350 mL of methanol and 158.1 g (1.0 mol) of 3-hydroxymethyl-5,5-dimethylhydantoin was added to a 1 L round-bottom flash. Then 106.2 g (1.0 mol) of diethanolamine (99%.purity) dissolved in 50 mL of methanol were added to the mixture dropwise with stirring at ambient temperature. After stirring at 75° C. for an additional 5 hours, the product was isolated and purified in the same manner as described in Example 1. The yield of the product was 84%. The product exhibited the same properties as those reported in Example 1. The same product could be made as an aqueous slurry using water as the solvent without the presence of methanol in less pure form, the solid being recovered by drying in air in near quantitative yield.

### Example 3

### Preparation of a Representative Chlorinated Heterocyclic Amine Diol: 1-cloro-3-[1-bis(N,N-2-hydroxyethyl) aminomethyl]-5,5-dimethyl-1,3-imidazolidin-2,4-dione

In this example, 5.0 g of the unhalogenated heterocyclic amine diol compound prepared as described in Example 1 were dissolved in 20 mL of distilled, deionized water in a 250 mL round-bottom flask. Then 10 mL of a 12.5% sodium hypochlorite solution were added, and the mixture was stirred at ambient temperature for 30 min. The product, 1-chloro-3-[1-bis(N,N-2-hydroxyethyl) aminomethyl]-5,5-dimethyl-1,3-imidazolidin-2,4-dione, was extracted out of the mixture using three 50 mL portions of chloroform. The extracts were combined and dried with 10 g of sodium sulfate for 4 hours at ambient temperature. After filtering out the sodium sulfate, the chloroform was removed using vacuum evaporation. The product was recovered as a clear, colorless oil. The product could also be made in chloroform solvent using t-butyl hypochlorite as the chlorinating agent.

### Example 4

### Biocidal Activity of the Chlorinated Heterocyclic Amine Diol: 1-chloro-3 [1-bis(N,N-2-hydroxyethyl) aminomethyl]-5,5-dimethyl-1,3-imidazolidin-2,4-dione

A 1957 mg/L solution of the chlorinated heterocyclic amine diol, prepared as described in Example 3 in chlorine-demand-free water at pH 7, was challenged with Staphylococcus aureus bacteria for contact times of 30 and 60 min at ambient temperature. Following the contact with the bacteria, further disinfectant action was quenched by adding 0.02 N sodium thiosulfate. Serial dilutions were then plated onto trypticase soy agar, and colony counts were made after 48 hours of incubation at 37° C. No growth was detected on the plates indicating a complete inactivation (>5 logs) at the two contact times. Thus the chlorinated compound is biocidal under the conditions tested.

### Example 5

### Preparation of a Biocidal Thin Film Protective Layer Using a Representative Heterocyclic Amine Diol: 3-[1-bis(N,N-2-hydroxyethyl) aminomethyl]-5,5-dimethyl-1,3-imidazolidin-2,4-dione

In this example, 0.7 g of the heterocyclic amine diol compound prepared as in Example 1 was added to 10.0 g of waterborne acrylic polyol with stirring until dissolution was complete. Then 2.45 g of isocyanate were thoroughly mixed in, followed by the addition and mixing of 2.10 g of distilled, deionized water. The procedure and proportions of constituents could be varied. For example, it was found that 1.0 g of the compound prepared as in Example 1 and 3.4 g isocyanate could be added to 10.0 g of waterborne acrylic polyol. After thorough mixing, 2.10 g distilled, deionized water were added and thoroughly mixed. The resulting formulations were immediately spread onto the surfaces of several plastic Petri dishes which were dried in air at ambient temperature. The thin films were dry to the touch within 4-5 hours, but were allowed to cure further overnight at ambient temperature before further treatment. The thin films prepared using the procedure and proportions described first above were then chlorinated by exposure to CLOROX bleach (5.25% sodium hypochlorite) at several concentrations for 3-12 hours. After rinsing thoroughly with chlorine-demand-free water, the thin films were dried in air for 6 hours and then analyzed for bound oxidative chlorine using an iodometric thiosulfate titration procedure. Other thin films prepared in the same manner at the same time (cut to squares of 6.45 cm² area) were challenged with Staphylococcus aureus for contact times of 2 hours. This was done by placing 25 μL of bacterial solution between two coated squares. Following quenching of disinfectant action with 0.02 N sodium thiosulfate in a vortexed solution in a beaker, serial dilutions of the vortexed solution were plated onto trypticase soy agar, incubated for 48 hours at 37° C., and colony counts were made. Unchlorinated thin films served as controls. The analytical and microbiological evaluations were performed as a function of chlorination concentration and of time following chlorination.

The data are shown in Tables 1 and 2. Table 1 shows that a complete inactivation of the bacteria (>4.5 logs) in 2 hours contact time was obtained after 10 and 5% CLOROX solutions were used for chlorination for 3 hours, and a 3.0 log inactivation occurred following exposure of the thin film to 1% CLOROX solution for 3 hours. This is consistent with the trend of Cl atoms/cm² for the identical samples. Table 2 shows that the thin films retained their biocidal efficacies for at least 14 days. It has also been demonstrated that biocidal efficacy can be regenerated once lost by reexposure to free chlorine solutions.

TABLE 1
THIN FILM CHLORINE LOADINGS AND BIOCIDAL
EFFICACIES AS A FUNCTION OF CHLORINATION
CONCENTRATION

| Clorox | | |
|---|---|---|
| Concentration % | Cl Atoms/cm² Surface | Log Reduction S. aureus |
| 10 | 1.34 × 10¹⁷ | >4.5 (no growth) |
| 5 | 9.13 × 10¹⁶ | >4.5 (no growth) |
| 1 | 3.69 × 10¹⁶⁶ | 3.0 |

TABLE 2
THIN FILM CHLORINE LOADINGS AND BIOCIDAL
EFFICACIES AS A FUNCTION OF TIME FOLLOWING
CHLORINATION WITH 100% CLOROX FOR 12 HOURS

| Time After Chlorination | | Log Reduction |
|---|---|---|
| in Days | Cl Atoms/cm² Surface | S. aureus |
| 0.25 | 3.53 × 10¹⁷ | >4.7 (no growth) |
| 4.0 | 6.78 × 10¹⁶ | >4.7 (no growth) |
| 14 | 2.33 × 10¹⁶ | >4.7 (no growth) |

Although the present invention has been described with reference to specific details of certain embodiments thereof, it is not intended that such details should be regarded as limitations upon the scope of the invention except as and to the extent that they are included in the accompanying claims. While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A compound, comprising:
a heterocyclic amine selected from at least one of a hydantoin, an imidazolidinone, an oxazolidinone, an isocyanurate, a glycoluril, or triazinedione; and
an amine polyol group covalently bonded to the heterocyclic amine through a linking group.

2. The compound of claim 1, wherein the amine polyol group is a diol.

3. The compound of claim 1, wherein the linking group is an alkylene comprising 1 to 6 carbons.

4. The compound of claim 1, wherein the linking group is methylene.

5. A compound having the formula: 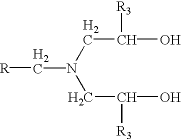
wherein R is a heterocyclic amine, the heterocyclic amine being at least one of a hydantoin, an imidazolidinone, an oxazolidinone, an isocyanurate, a glycoluril, or triazinedione; and
wherein R₃ is at least one of hydrogen and an alkyl group of 1 to 6 carbons.

6. A compound having the formula: 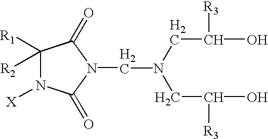
wherein R₁ and R₂ are at least one of an alkyl group of 1 to 6 carbons or phenyl group;
wherein R₃ is at least one of hydrogen or an alkyl group of 1 to 6 carbons; and
wherein X is at least one of hydrogen or a halogen.

7. The compound of claim 6, wherein R₁ and R₂ are methyl and R₃ and X are hydrogen.

8. The compound of claim 6, wherein R₁ and R₂ are methyl; R₃ is hydrogen; and X is chlorine.

9. The compound of claim 6, wherein R₁ and R₂ are methyl; R₃ is hydrogen; and X is bromine.

10. A method of making a compound having the formula: 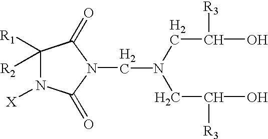
wherein R₁ and R₂ are at least one of an alkyl group of 1 to 6 carbons or phenyl group;
wherein R₃ is at least one of hydrogen or an alkyl group of 1 to 6 carbons; and
wherein X is hydrogen;
comprising reacting a 5,5-dialkylhydantoin with formaldehyde and an aminoalcohol.

11. The method of claim 10, wherein the 5,5-dialkylhydantoin is 5,5-dimethylhydantoin and the aminoalcohol is diethanolamine.

12. A method of making a compound having the formula: 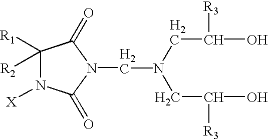
wherein R₁ and R₂ are at least one of an alkyl group of 1 to 6 carbons or phenyl group;
wherein R₃ is at least one of hydrogen or an alkyl group of 1 to 6 carbons; and
wherein X is hydrogen;
comprising reacting a 3-hydroxyalkyl-5,5-dialkylhydantoin with an aminoalcohol.

13. The method of claim 12, wherein the 3-hydroxyalkyl-5,5-dialkylhydantoin is 3-hydroxymethyl-5,5-dimethylhydantoin and the aminoalcohol is diethanolamine.

14. A composition, comprising at least one compound having the formula: 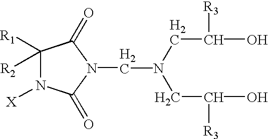
wherein R₁ and R₂ are at least one of an alkyl group of 1 to 6 carbons or phenyl group;
wherein R₃ is at least one of hydrogen or an alkyl group of 1 to 6 carbons; and
wherein X is at least one of hydrogen or a halogen.

15. The composition of claim 14, wherein R₁ and R₂ are methyl and R₃ and X are hydrogen.

16. The composition of claim 14, wherein R₁ and R₂ are methyl; R₃ is hydrogen; and X is chlorine.

17. The composition of claim 14, wherein R₁ and R₂ are methyl; R₃ is hydrogen; and X is bromine.

18. The composition of claim 14, further comprising an acrylic polyol.

19. The composition of claim 14, further comprising at least one resin selected from epoxides, polyurethanes, polyureas, acrylics, vinyls, silanes, and siloxanes.

20. A polymer, made by the process comprising mixing at least one compound having a functional group reactive toward an alcohol group and at least one compound having the formula: 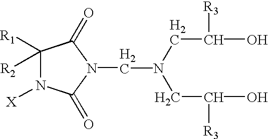
wherein R₁ and R₂ are at least one of an alkyl group of 1 to 6 carbons or phenyl group;
wherein R₃ is at least one of hydrogen or an alkyl group of 1 to 6 carbons; and
wherein X is at least one of hydrogen or a halogen.

21. The polymer of claim 20, wherein R₁ and R₂ are methyl and R₃ and X are hydrogen.

22. The polymer of claim 20, wherein R₁ and R₂ are methyl; R₃ is hydrogen; and X is chlorine.

23. The polymer of claim 20, wherein R₁ and R₂ are methyl; R₃ is hydrogen; and X is bromine.

24. The polymer of claim 20, wherein the compound with functional group reactive toward an alcohol group is an isocyanate.

25. The polymer of claim 20, wherein the compound with functional group reactive toward an alcohol group is an isocyanate; and wherein R₁ and R₂ are methyl; wherein R₃ is hydrogen; and wherein X is chlorine.

26. The polymer of claim 20, wherein the compound with functional group reactive toward an alcohol group is an isocyanate; and wherein R₁ and R₂ are methyl; wherein R₃ is hydrogen; and wherein X is bromine.

27. A method for making a polymer, comprising:
mixing at least one compound having a functional group reactive toward an alcohol group and at least one compound having the formula: 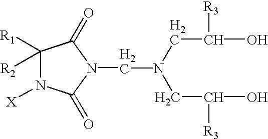
wherein R₁ and R₂ are at least one of an alkyl group of 1 to 6 carbons or phenyl group;
wherein R₃ is at least one of hydrogen or an alkyl group of 1 to 6 carbons; and
wherein X is at least one of hydrogen or a halogen; and
curing the mixture to form a polymer.

28. The method of claim 27, wherein R₁ and R₂ are methyl and R₃ and X are hydrogen.

29. The method of claim 28, further comprising exposing the polymer to halogen to render the polymer biocidal.

30. The method of claim 29, wherein R₁ and R₂ are methyl; R₃ is hydrogen; and X is chlorine.

31. The method of claim 29, wherein R₁ and R₂ are methyl; R₃ is hydrogen; and X is bromine.

32. The method of claim 30, wherein the compound with functional group reactive toward an alcohol group is an isocyanate.

33. A method for making a thin film protective layer, comprising:
mixing at least one compound having a functional group reactive toward an alcohol group; and
at least one compound having the formula: 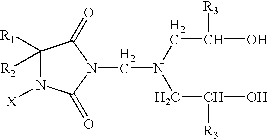
wherein R₁ and R₂ are at least one of an alkyl group of 1 to 6 carbons or phenyl group;
wherein R₃ is at least one of hydrogen or an alkyl group of 1 to 6 carbons; and
wherein X is at least one of hydrogen or a halogen;
forming a thin layer from the mixture; and
allowing the thin layer to cure.

34. The method of claim 33, wherein the compound with functional group reactive toward an alcohol is an isocyanate.

35. The method of claim 33, wherein R₁ and R₂ are methyl and wherein R₃ and X are hydrogen.

36. The method of claim 35, further comprising exposing the thin layer to halogen to render the thin layer biocidal.

37. The method of claim 36, wherein the halogen is at least one of chlorine or bromine.

38. The method of claim 33, comprising charging the thin layer with a charging solution comprising a halogen.

39. The method of claim 38, comprising recharging the thin layer with a halogen.

40. The method of claim 39, where the thin layer is recharged with a charging solution comprising a halogen.

41. A paint, comprising a paint component having a heterocyclic amine group covalently bonded to a urethane group through an amine polyol group.

42. The paint of claim 41, wherein the amine polyol group is a diethanol amine group.

43. The paint of claim 41, wherein the heterocyclic amine is at least one of a hydantoin, an imidazolidinone, an oxazolidinone, an isocyanurate, a glycoluril, or triazinedione.

44. The paint of claim 41, wherein the heterocyclic amine group comprises a halogen bonded at a nitrogen heteroatom.

45. A paint made by the process comprising, reacting paint component having an isocyanate group with a compound having the formula: 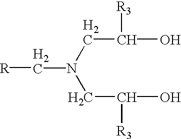
wherein R is a heterocyclic amine, the heterocyclic amine being at least one of a hydantoin, an imidazolidinone, an oxazolidinone, an isocyanurate, a glycoluril, or triazinedione; and
R₃ is at least one of hydrogen and an alkyl group of 1 to 6 carbons.

46. A paint made by the process, comprising reacting paint component having an isocyanate group with a compound having the formula: 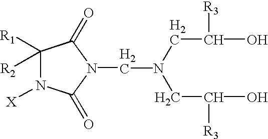
wherein R₁ and R₂ are at least one of an alkyl group of 1 to 6 carbons or phenyl group;
wherein R₃ is at least one of hydrogen or an alkyl group of 1 to 6 carbons; and
wherein X is at least one of hydrogen or a halogen.

47. A polymer, comprising moieties having the formula: 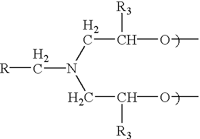
wherein R is a heterocyclic amine from at least one of a hydantoin, an imidazolidinone, an oxazolidinone, an isocyanurate, a glycoluril, or triazinedione; and
wherein R₃ is at least one of hydrogen and an alkyl group of 1 to 6 carbons.

48. The polymer of claim 47, wherein the polymer is at least one from epoxides, polyurethanes, polyureas, acrylics, vinyls, silanes, and siloxanes.

49. A polymer, comprising moieties having the formula: 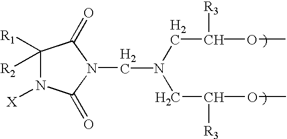
wherein R₁ and R₂ are at least one of an alkyl group of 1 to 6 carbons or phenyl group;
wherein R₃ is at least one of hydrogen or an alkyl group of 1 to 6 carbons; and
wherein X is at least one of hydrogen or a halogen.

50. The polymer of claim 49, wherein the polymer is at least one from epoxides, polyurethanes, polyureas, acrylics, vinyls, silanes, and siloxanes.

51. A polyurethane polymer, comprising moieties having the formula: 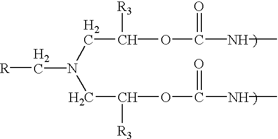
wherein R is a heterocyclic amine from at least one of a hydantoin, an imidazolidinone, an oxazolidinone, an isocyanurate, a glycoluril, or triazinedione; and
wherein R₃ is at least one of hydrogen and an alkyl group of 1 to 6 carbons.

52. A polyurethane polymer, comprising moieties having the formula: 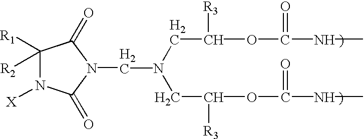
wherein R₁ and R₂ are at least one of an alkyl group of 1 to 6 carbons or phenyl group;
wherein R₃ is at least one of hydrogen and an alkyl group of 1 to 6 carbons; and
wherein X is at least one of hydrogen or a halogen.

53. An article, comprising moieties having the formula: 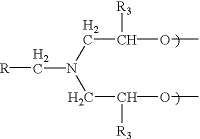
wherein R is a heterocyclic amine from at least one of a hydantoin, an imidazolidinone, an oxazolidinone, an isocyanurate, a glycoluril, or triazinedione; and
wherein R₃ is at least one of hydrogen and an alkyl group of 1 to 6 carbons.

54. The article of claim 53, wherein the article is one of at least a sponge and a foam.

55. An article, comprising moieties having the formula: 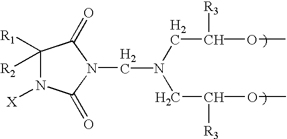
wherein R₁ and R₂ are at least one of an alkyl group of 1 to 6 carbons or phenyl group;
wherein R₃ is at least one of hydrogen or an alkyl group of 1 to 6 carbons; and
wherein X is at least one of hydrogen or a halogen.

56. The article of claim 55, wherein the article is one of at least a sponge and a foam.
